(19) United States
(12) Patent Application Publication
(10) Pub. No.: US 2008/0317830 A1
(43) Pub. Date: Dec. 25, 2008

(54) Compositions and Methods for the Treatment of Wounds and Scar Tissue

(75) Inventors: Jay A. Goldstein, Newton, MA (US); Bernard Grubstein, Wayland, MA (US); Michael Rothman, Newton, MA (US); Whe-Yong Lo, North Attleboro, MA (US); Patrea L. Pabst, Dewey Rose, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: LioLabs LLC, Newton, MA (US)

(21) Appl. No.: 11/768,053

(22) Filed: Jun. 25, 2007

(51) Int. Cl. A61K 33/24; A61K 33/08; A61K 33/30; A61L 15/18; A61K 33/12

(52) U.S. Cl. 424/447; 424/617; 424/642; 424/683; 424/689

(57) ABSTRACT

Compositions for the treatment of wounds and/or scars are described herein. The compositions contain between 1 and up to 30% by weight, more preferably between 1 and 20%, most preferably between about 5 and 10% by weight particles, such as titanium dioxide or a similar material in a pharmaceutically acceptable base or carrier, such as petrolatum. The compositions are less greasy than petrolatum alone, and thus are more aesthetically pleasing. The compositions exhibit occlusive properties comparable to petrolatum. The compositions are absorbed into the skin, unlike petrolatum, and exhibit significant wound healing characteristics not observed with petrolatum alone. In one embodiment, the pharmaceutically acceptable base is petrolatum and the particles are titanium dioxide. The compositions can be used to treat complex, hard to heal wounds, such as diabetic ulcers; pressure sores, such as bed sores; lacerations; bite wounds; burns; penetrating wounds; surgical wounds, etc. The composition can also be used to promote normal healing of scar tissue. The compositions can also be used for the topical delivery of one or more active agent. The compositions can be used to reduce fine lines and wrinkles, and to rehydrate skin or to treat dry skin.

### FIELD OF THE INVENTION

The present invention is in the field of pharmaceutical compositions, particularly compositions for the treatment of wounds and/or scars.

### BACKGROUND OF THE INVENTION

Petrolatum, also known as petroleum jelly, is a semi-solid mixture of hydrocarbons, originally promoted as a topical ointment for its sealing, or occlusive, properties. Petrolatum is colorless, or of a pale yellow color (when not highly distilled), translucent, and devoid of taste and smell when pure. It does not oxidize on exposure to the air, and is not readily acted on by chemical reagents. It is insoluble in water. It is soluble in chloroform, benzene, carbon disulfide and oil of turpentine. Petroleum jelly is sold under the trademark Vaseline®.

Vaseline® was originally promoted as an ointment for scrapes, burns, and cuts. Physicians have shown, however, that Vaseline® has no medicinal effect or any effect on the blistering process, nor is it absorbed by the skin. Vaseline's effectiveness in accelerating wound healing is believed to stem from its sealing, or occlusive, effect on cuts and burns, which inhibits germs from getting into the wound and keeps the injured area supple by preventing the skin's moisture from evaporating.

Vaseline® has several drawbacks. Vaseline® is a very greasy material. It collects dirt at the site of application because it is not absorbed. It is removed by application of a bandage or washing.

It is therefore an object of the invention to provide compositions which promote wound healing, wherein the compositions, unlike petrolatum, are absorbed into the skin, and are less greasy than petrolatum.

It is further an object of the invention to provide a delivery vehicle for the topical administration of one or more active agents, wherein the delivery vehicle is less greasy than petrolatum.

It is still further an object of the invention to provide compositions which can be used to reduce fine lines and wrinkles, rehydrate the skin and which are less greasy than Petrolatum.

### SUMMARY OF THE INVENTION

Compositions for the treatment of wounds and/or scars and as a hydrating agent are described herein. The compositions contain between 1 and up to 30% by weight, more preferably between 1 and 20%, most preferably between about 5 and 10% by weight particles, such as titanium dioxide or a similar material in a petrolatum pharmaceutically acceptable base or carrier. The compositions are less greasy than petrolatum alone, and thus are more aesthetically pleasing. The compositions exhibit occlusive properties comparable to petrolatum. The compositions are absorbed into the skin, unlike petrolatum, and exhibit significant wound healing characteristics not observed with petrolatum alone. In one embodiment, the pharmaceutically acceptable base is petrolatum and the particles are titanium dioxide.

The compositions can be used to treat complex, hard to heal wounds, such as diabetic ulcers; pressure sores, such as bed sores; lacerations; bite wounds; burns; penetrating wounds; surgical wounds, etc. The composition can also be used to promote normal healing of scar tissue. The compositions can also be used for the topical delivery of one or more active agent. The compositions can be used to reduce fine lines and wrinkles, and to rehydrate skin or to treat dry skin.

### DETAILED DESCRIPTION OF THE INVENTION

### I. Definitions

“Base” or “carrier”, as used herein, refers to a pharmaceutically acceptable material useful for delivering one or more active agents. The active agents can be dissolved or dispersed in the base or carrier. In one embodiment, the base or carrier is one suitable for topical administration of active agents.

“Wound”, as used herein, refers to a break in the integrity of the skin. Examples of wounds include, but are not limited to, diabetic ulcers; pressure sores, such as bed sores; lacerations; bite wounds; burns; penetrating wounds; surgical wounds, etc.

“Occlusiveness”, as used herein, refers to the ability of a material to prevent the evaporative loss of water from the skin.

“Active agent”, as used herein, refers to a therapeutic, prophylactic, or diagnostic agent.

“Particles”, as used herein, refers to particles having a diameter less than about fifty microns. For example, the particles can have a diameter between 0.01 and 50 microns, preferably between 0.01 and 25 microns, more preferably between 0.01 and 10 microns.

### II. Compositions

Compositions for the treatment of wounds or scar tissue, for rehydrating of normal skin, and/or for the topical delivery of one or more active agents are described herein. The compositions contain particles, such as titanium dioxide or a similar material in a pharmaceutically acceptable base or carrier, such as petrolatum. The compositions are absorbed into the skin, unlike petrolatum, and exhibit significant wound healing characteristics not observed with petrolatum alone.

A. Particles

The compositions contain particles such as titanium dioxide; zinc oxide, such as an ultrafine grade of micronized zinc oxide, magnesium oxide, talc, and combinations thereof. In the preferred embodiment, the particles are titanium dioxide.

Titanium dioxide, also known as titanium(IV)oxide or titania, is the naturally occurring oxide of titanium. The chemical formula of titanium dioxide is TiO₂. Titanium dioxide occurs in four forms: rutile, a tetragonal mineral usually of prismatic habit, often twinned; anatase or octahedrite, a tetragonal mineral of dipyramidal habit; brookite, an orthorhombic mineral; and titanium dioxide (B) or TiO₂(B), a monoclinic mineral. Anatase and brookite are relatively rare minerals.

As a pigment of high refringence, titanium dioxide is the most widely used white pigment because of its brightness and very high refractive index (n=2.4). When deposited as a thin film, the color and refractive index of titanium dioxide make it an excellent reflective optical coating for dielectric mirrors and some gemstones, for example “mystic fire topaz”. TiO₂ is also an effective opacifier in powder form, where it is employed as a pigment to provide whiteness and opacity to products such as paints, coatings, plastics, papers, inks, foods, and most toothpastes. In cosmetic and skin care products, titanium dioxide is used both as a pigment and a thickener.

Titanium dioxide is found in almost every sunscreen with a physical blocker both because of its refractive index and its resistance to discoloration under ultraviolet light. This advantage enhances its stability and ability to protect the skin from ultraviolet light. Sunscreens designed for infants or people with sensitive skin are often based on titanium dioxide and/or zinc oxide, as these mineral UV blockers are less likely to cause skin irritation than chemical UV absorber ingredients, such as avobenzone. However, sunscreens require a high concentration of titanium dioxide in order to be effective, typically 30% or greater.

The compositions contain titanium dioxide, or a similar material, at a concentration of between about 1% and up to about 30%, more preferably between 1 and 20% by weight, most preferably between 5 and 10% by weight of the composition. The titanium dioxide used to prepare the formulations generally has a diameter less than about 100 microns, preferably between 10 nm and 100 microns. In one embodiment, the diameter of the titanium dioxide particles is 44 microns. In another embodiment, the diameter of the titanium dioxide particles is 0.3 microns. In still another embodiment, the diameter of the titanium dioxide particles is 15 nm (0.015 microns).

B. Base or Carrier

The titanium dioxide is dissolved or dispersed in a pharmaceutically acceptable carrier or base. Suitable bases include, but are not limited to, petrolatum, mineral oil, vegetable oils, and combinations thereof. In the preferred embodiment, the base is petrolatum.

Petrolatum, also known as petroleum jelly, is a semi-solid mixture of hydrocarbons, originally promoted as a topical ointment for its sealing, or occlusive, properties. It's claimed medicinal value has since been limited by certain health and safety concerns. Petroleum jelly is sold under the trademark Vaseline®. Petrolatum is a flammable, semi-solid mixture of hydrocarbons, having a melting-point usually ranging from a little below to a few degrees above 100° F. (37° C.). It is colorless, or of a pale yellow color (when not highly distilled), translucent, and devoid of taste and smell when pure. It does not oxidize on exposure to the air, and is not readily acted on by chemical reagents. It is insoluble in water. It is soluble in chloroform, benzene, carbon disulfide and oil of turpentine.

Petrolatum was originally promoted as an ointment for scrapes, burns, and cuts. Physicians, however, have shown that Petrolatum has no medicinal effect or any effect on the blistering process, nor is it absorbed by the skin. Petrolatum's effectiveness in accelerating wound healing stems from its sealing, or occlusive, effect on cuts and burns, which inhibits germs from getting into the wound and keeps the injured area supple by preventing the skin's moisture from evaporating.

In contrast, as demonstrated by the examples, the compositions containing the titanium particles are absorbed into the skin and exhibit wound healing characteristics not observed with petrolatum alone. The compositions are also less greasy and exhibit reduced tackiness compared to petrolatum alone.

C. Active Agents

The compositions can also be used for the topical administration of one or more active agents. Suitable classes of active agents include, but are not limited to, antimicrobial agents, such as antibacterial agents, antibiotics, antiviral agents, and antifungal agents; topical steroids; topical anesthetics; anti-inflammatory agents; and combinations thereof.

The anti-inflammatory agent can be a corticosteroid or a non-steroidal anti-inflammatory drug (NSAID). Suitable corticosteroids include alclometasone dipropionate, amcinonide, beclamethasone dipropionate, betamethasone benzoate, betamethasone dipropionate, betamethasone valerate, budesonide, clobetasol propionate, clobetasone butyrate, desonide, desoxymethasone, diflorasone diacetate, diflucortolone valerate, flumethasone pivalate, fluclorolone acetonide, fluocinolone acetonide, fluocionoide, fluocortin butyl, flucortolones, fluprednidene acetate, flurandrenolone, halcinonide, hydrocortisone, hydrocortisone acetate, hydrocortisone butyrate, methylprednisolone acetate, nometasone furoate, triamcinolone acetonide, and de-esterified base compounds, esters of base compounds, salts thereof and combinations thereof.

Suitable NSAIDs include diclofenac, ibuprofen, acetylsalicylic acid, piroxicam, ketoprofen, felbinac, and benzylamine. Such NSAIDs may be present with or without a hydrocortisone-type anti-inflammatory.

Suitable anesthetics include the aminoacylanilide compounds such as lidocaine, prilocalne, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

Suitable antifungal agents include clotrimazole, econazole, ketoconazole, itraconazole, miconazole, oxiconazole, sulconazole, butenafine, naftifine, terbinafine, undecylinic acid, tolnaftate, nystatin, and sertaconazole nitrate. Any conventional topical antibiotic can be used; for example, the antibacterial agent fusidic acid. Suitable antiviral agents include, but are not limited to, acyclovir.

As used herein, “pharmaceutically acceptable salts” refer to derivatives of the disclosed compounds wherein the parent compound is modified by making acid-addition or base-addition salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

D. Excipients and Additives

The compositions may contain one or more pharmaceutically acceptable excipients and/or additives. Suitable excipients and/or additives include, but are not limited to, pH modifying agents, preservatives, antioxidants, humectants, chelating agents, suspending agents, thickening agents, stabilizing agents, solubilizing agents, penetration enhancers, and combinations thereof. In the preferred embodiment the formulation contains only petrolatum and titanium particles

E. Bandages

The formulation can be administered to the surface of an injury which is left uncovered or bandaged. The formulation can also be incorporated into the bandage. For example, the formulation can be applied to a bandage, gauze, cotton pads, strips, Q-tips, or pressure bandages. The formulation can be incorporated into the bandage or applied to the surface. The surface application may be protected with a plastic film or membrane to keep it clean and in place. The titanium particles have hemostatic properties so the composition is particularly effective in combination with bandages applied to sites of bleeding.

In addition to the formulation which contains titanium dioxide which is a known hemostatic agent, the bandage can also be impregnated with one or more materials which promote clotting. For example, calcium ions (Ca²⁺) are known to aid in clotting. A calcium ion source can be impregnated into the bandage. Suitable calcium ion sources include, but are not limited to, inorganic calcium salts, such as calcium carbonate, and organic calcium salts, such as calcium salts of one or more active agents, such as antibiotic agents.

### III. Methods of Making

The formulations are prepared using standard techniques to blend the particles into the petrolatum base. The compositions can be prepared as an ointment. Ointments typically contain an oil-base (e.g., petrolatum) and thus are water insoluble. In contrast, creams and lotions typically contain one or more surfactants which makes them water soluble.

### IV. Method of Using

A. Healing of Wounds and Scar Tissue

The compositions can be used for the treatment of a wide variety of wounds, particularly complex and difficult to heal wounds. Examples of such wounds include, but are not limited to, diabetic ulcers; pressure sores, such as bed sores; lacerations; bite wounds; burns; penetrating wounds; surgical wounds, etc. The compositions are administered in an amount effective to promote healing of the wound. The compositions can be administered alone or in combination with a bandage. For example, for veterinary applications, the compositions typically would be administered in combination with a bandage in order to keep dirt and debris away from the wound.

As demonstrated by the examples, the combination of the titanium particles and petrolatum enhance the rate of healing and restore a more normal histology to injured healed tissue. The mechanism is not known but it is possible this is due to the titanium particles attracting phagocytic cells in to the injured area.

B. Topical Vehicle for Drug Delivery

The titanium dioxide/petrolatum formulations described herein can be used as a vehicle for the topical administration one or more active agents. The active agent can be dissolved or dispersed within the particle/petrolatum formulations. In addition to the active agent(s), the formulations may contain one or more pharmaceutically acceptable excipients.

C. Treatment of Lines and Wrinkles and Rehydration of Skin

The titanium dioxide/petrolatum formulations d can also be used to treat fine lines and wrinkles. It is believed that the compositions are effective at treating fine lines and wrinkles by decreasing or eliminating transepidermal water loss. The composition leaves a minimal greasy residue and is thus more aesthetically pleasing than Petrolatum. The compositions described are absorbed into human skin, unlike petrolatum.

Laser therapy is used to treat a variety of disorders, such as wrinkles, age spots, and acne. Many of these procedures require the skin to be moisturized after the procedure. The compositions can be used to treat dry skin that results from laser therapy without the greasiness associated with Petrolatum.

D. Impregnated Bandages

Bandages impregnated with Petrolatum have been used for wound protection and hydration. Petrolatum, however, is greasy and tacky, making the product difficult to handle and apply. This is particularly true for the treatment of nosebleeds and/or nose surgeries, such as sinus surgery. The non-greasy formulations described herein can be applied to, or impregnated into, any of the same materials: gauze, bandaids, foams, wraps, drapes, etc. to provide the same non-stick occlusiveness in combination with more absorbence into the wound, less greasiness, and promotion of wound healing.

The present invention will be further understood by reference to the following non-limiting examples.

### EXAMPLES

### Example 1

### Wound Healing Formulation Containing Titanium Dioxide and Petrolatum for Treatment of Chronic Non-Healing Wound

Materials and Methods

Samples were prepared containing 1%, 5% and 10% titanium particles homogenously blended into petrolatum.

An eight year old Arabian horse having a chronic non-healing injury to the right rear hock was treated for a period of two weeks. The horse had put its hoof through a sheet of galvanized metal and removed a full thickness layer of dermis, into the tendons and muscle around the hock, approximately three-fourths of the way around the hock. After four months of treatment, approximately one-fourth of the initial injury remained unhealed, with a large amount of unpigmented granulation tissue that bled frequently and was scabbed over. This was unchanged after an additional 2-3 months.

To the right half of the injury Vaseline® was applied and 10% titanium formulation was applied to the left half, twice a day, with rubbing, for a period of two weeks.

The injury was then left untreated for one month. Then 5% titanium formulation was applied to the entire injured area, approximately once a day for 2-3 weeks.

Results and Observations

The Vaseline® sits on the surface of the injury. The titanium formulation penetrates and provides a moisturing effect much longer than the Vaseline®.

The results during the first two weeks of treatment were extremely surprising. The treated injury began healing, even though there had been almost no healing in several months. There was a migration into the area of normal epithelial cells, the skin became black, and over time some hair follicles have appeared. The Vaseline® treated area showed no change.

In the second treatment, where the area previously treated only with Vaseline®, was also treated with the titanium formulation, normal epithelial cells began to migrate into the injured area, and the tissue increased in pigmentation. The scabbed over area, which had not changed in composition for several months, began to heal.

At the conclusion of the study the injury that had been treated with the titanium formulation initially was almost completely healed; the control then treated injury was healing rapidly. No difference was observed between the 5% and 10% formulations.

### Example 2

### Removal of Scar Tissue Using Formulation Containing Titanium Dioxide and Petrolatum

Materials and Methods

Samples were prepared containing 1%, 5% and 10% titanium particles homogenously blended into petrolatum.

A 21 year old Arabian stallion with a large amount of fibrotic and hard, shiny scar tissue on his right rear leg was treated to remove scar tissue and to see if the formulation could induce normal healing of an old scar. The scar tissue extended over an area of approximately one inch by eight inches, and was up to an inch thick in one region.

The 10% titanium formulation was applied to the scar tissue, the leg wrapped in gauze, then cotton batting, then wrapped tightly with self-adhesive elastic wrap. This was changed approximately every 5 to 7 days over a period of one month. The treatment was then discontinued.

Results

After only two weeks, all of the scar tissue had “dissolved” and could be removed by wiping with the cotton gauze. The normal but unpigmented and hairless tissue underneath appeared pink and vascularized instead of white and shiny. After another two weeks, the width of the scar tissue had narrowed slightly, with ingrowth from the adjacent epithelium.

Treatment was discontinued and no further healing was observed.

### Example 3

### Measurement of the Trans-Epidermic Water Loss (TEWL) of the Titanium Dioxide/Petrolatum Formulation Compared to Petrolatum Alone

Occlusiveness of the formulations described herein was evaluated using transepi-water loss (TEWL). TEWL was measured using a Tewameter® TM 300. The Tewameter® contains a probe which can be placed on the surface of the skin for the TEWL measurement.

Measurement Conditions

The Tewameter® was set to calculate and display average TEWL (g/hm²) and standard deviation (SD, g/hm²) at ten second intervals. The duration of the measurement was 60 seconds. The average TEWL was recorded at 30 seconds and 60 seconds. A “selected” average value, which was selected by the Tewameter® software program at the time point at which the SD was at its minimum during the 60 second measurement, was displayed at the end of the measurement. The average TEWL measurements at 30 seconds, 60 seconds, and the “selected” value were used for evaluation of the occlusiveness of the topical formulations.

Study Design

Four formulations were evaluated: (1) petrolatum (Vaseline®), referred to as Formulation A; (2) 1% titanium dioxide in Vaseline®, referred to as Formulation B; (3) 5% titanium dioxide in Vaseline®, referred to as Formulation C; and (4) 10% titanium dioxide in Vaseline®, referred to as Formulation D.

Four locations on the right and left forearm of the study participants were used for the study. On each testing day, TEWL was measured for each of the eight sites prior to application of the formulation. The formulation to be studied was then applied to each of the eight sites. The formulation was rubbed into the skin to form a thin layer prior to TEWL measurement. The amount of the formulation applied and the amount of time rubbing the formulation into the skin were consistent for each application.

The formulations were applied in a four-way cross over fashion on each arm with the four sequences shown below. A washout period of one day between study periods was observed.

| Site | 2 | 3 | 4 | 5 |
|---|---|---|---|---|
| Sequence 1 (S1) | A | B | C | D |
| Sequence 2 (S2) | B | A | D | C |
| Sequence 3 (S3) | C | D | A | B |
| Sequence 4 (S4) | D | C | B | A |

Two volunteers were enrolled in the study. For each volunteer, two readings (one from each arm) were collected for each formulation tested for a total of 8 measurements per formulation. Upon completion of the four sequences, a total of eight (8) readings were collected for each formulation from each volunteer.

The mean TEWL were used to evaluate occlusiveness; the smaller the TEWL, the more occlusive the formulation. The mean and SD TEWL at 30 seconds, 60 seconds, and the “selected TEWL” were calculated from the eight readings for each formulation obtained from each volunteer. The mean and SD TEWL at 30 seconds, 60 seconds, and the “selected TEWL” were also calculated from the 16 readings for each formulation obtained from the two volunteers. The mean baseline TEWL was also calculated for each application site for prior and post-application TEWL comparison. The results are shown in the tables below.

Tables 1 and 2 show the TEWL data collected from a four-way cross over experiment (4 sequences S1 to S4) on both forearms for subject WL without and with baseline correction, respectively.

Tables 3 and 4 show the TEWL data collected from a four-way cross over experiment (4 sequences S1 to S4) on both forearms for subject WL without and with baseline correction, respectively.

Table 5 shows the mean and SD TEWL at 30 seconds, 60 seconds, and the “selected TEWL” calculated from the 16 readings for each formulation obtained from the two volunteers.

Table 6 shows the ratio of TEWL of the Petrolatum/Titanium Oxide Formulations to Petrolatum for subject WL (mean of 8 measurements), SL (mean of 8 measurements), and WL and SL (mean of 16 measurements).

The data shows that the petrolatum/titanium dioxide formulations were at least as occlusive as Vaseline®. However, the petrolatum/titanium dioxide formulations are less greasy than Vaseline® and thus more aesthetically pleasing.

### Example 4

### Application of Titanium Formulation to Normal Human Skin

The formulation containing 5% titanium dioxide was applied to normal skin. It was well received, felt to be cool, non-greasy and when washed off left the skin well hydrated.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be compassed by the following claims.

TABLE 1
Occlusiveness data for test subject WL

| | Petrolatum | | | 1% TiO₂ | | | 5% TiO₂ | | | 10% TiO₂ | | |
|---|---|---|---|---|---|---|---|---|---|---|---|---|
| Site | 30 sec | 60 sec | Selected | 30 sec | 60 sec | Selected | 30 sec | 60 sec | Selected | 30 sec | 60 sec | Selected |
| S1-L | 2.83 | 1.77 | 2.19 | 1.12 | 0.5 | 0.95 | 2.78 | 2.37 | 2.71 | 2.33 | 1.92 | 2.07 |
| S1-R | 2.29 | 2.01 | 1.99 | 3.18 | 2.62 | 3.07 | 3.5 | 2.7 | 2.72 | 2.77 | 2.16 | 2.34 |
| S2-L | 2.5 | 1.75 | 2.03 | 2.92 | 2.08 | 2.58 | 3.36 | 3.07 | 3.35 | 4.38 | 3.47 | 5.9 |
| S2-R | 3.92 | 3.04 | 3.25 | 4.07 | 3.39 | 3.97 | 3.88 | 3.11 | 4.41 | 4.84 | 4.19 | 4.2 |
| S3-L | 4.2 | 3.29 | 3.32 | 4.24 | 3.69 | 4 | 4.24 | 3.3 | 3.96 | 4.4 | 4.2 | 4.39 |
| S3-R | 4.5 | 4.24 | 4.45 | 3.8 | 3.27 | 4.39 | 2.64 | 1.93 | 3.04 | 3.8 | 3.29 | 3.38 |
| S4-L | 2.45 | 1.98 | 2.37 | 4.38 | 3.84 | 5.14 | 1.5 | 1.84 | 1.3 | 1.59 | 1.03 | 1.03 |
| S4-R | 2.14 | 1.54 | 2.19 | 2.04 | 1.88 | 1.66 | 3.37 | 2.16 | 3 | 3.25 | 2.21 | 3.12 |
| Mean (N = 8) | 3.10 | 2.45 | 2.72 | 3.22 | 2.66 | 3.22 | 3.16 | 2.56 | 3.06 | 3.42 | 2.81 | 3.30 |
| SD | 0.95 | 0.96 | 0.87 | 1.16 | 1.13 | 1.43 | 0.85 | 0.57 | 0.93 | 1.14 | 1.15 | 1.53 |
| RSD, % | 30.5 | 39.1 | 32.0 | 35.9 | 42.6 | 44.3 | 26.9 | 22.1 | 30.4 | 33.2 | 40.9 | 46.3 |

TABLE 2
Occlusiveness data for subject SL

| | Petrolatum | | | 1% TiO₂ | | | 5% TiO₂ | | | 10% TiO₂ | | |
|---|---|---|---|---|---|---|---|---|---|---|---|---|
| Site | 30 sec | 60 sec | Selected | 30 sec | 60 sec | Selected | 30 sec | 60 sec | Selected | 30 sec | 60 sec | Selected |
| S1-L | 0.2 | −0.76 | −0.69 | 1.13 | 0.93 | 1.0 | 2.51 | 1.85 | 1.85 | 3.14 | 3.49 | 3.06 |
| S1-R | 4.32 | 3.64 | 3.87 | 3.13 | 2.15 | 2.72 | 3.92 | 3.52 | 3.52 | 3.9 | 2.92 | 3.78 |
| S2-L | 2.42 | 1.68 | 1.91 | 3.01 | 1.8 | 1.96 | 4.75 | 3.73 | 4.27 | 3.22 | 2.27 | 2.56 |
| S2-R | 5.58 | 5.12 | 5.18 | 4.28 | 3.85 | 4.3 | 4.29 | 3.65 | 4.17 | 4.32 | 4.4 | 4.22 |
| S3-L | 2.08 | 1.72 | 1.72 | 3.59 | 3.03 | 3.06 | 1.83 | 0.85 | 1.63 | 2.34 | 1.27 | 1.86 |
| S3-R | 4.06 | 3.75 | 4.06 | 3.8 | 2.5 | 3.93 | 3.65 | 3.57 | 3.52 | 2.85 | 1.42 | 3.06 |
| S4-L | 4.65 | 4.16 | 4.2 | 3.69 | 3.05 | 3.55 | 4.5 | 3.71 | 4.21 | 2.66 | 1.43 | 1.91 |
| S4-R | 3.08 | 2.67 | 2.91 | 3.74 | 3.44 | 3.35 | 3.76 | 3.21 | 3.29 | 4.7 | 4.09 | 4.63 |
| Mean (N = 8) | 3.30 | 2.75 | 2.90 | 3.30 | 2.59 | 2.98 | 3.65 | 3.01 | 3.31 | 3.39 | 2.66 | 3.14 |
| SD | 1.71 | 1.85 | 1.87 | 0.96 | 0.95 | 1.08 | 1.00 | 1.07 | 1.04 | 0.83 | 1.25 | 1.02 |
| RSD, % | 52.0 | 67.3 | 64.5 | 29.1 | 36.6 | 36.1 | 27.5 | 35.6 | 31.3 | 24.6 | 47.0 | 32.6 |

TABLE 3
Post-dosing data from a four-way crossover (4 sequences) for subjects WL and SL

| | Petrolatum | | | 1% TiO₂ | | | 5% TiO₂ | | | 10% TiO₂ | | |
|---|---|---|---|---|---|---|---|---|---|---|---|---|
| | 30 | 60 | | 30 | 60 | | 30 | 60 | | 30 | 60 | |
| Site | sec | sec | Selected* | sec | sec | Selected* | sec | sec | Selected* | sec | sec | Selected* |
| S1-L-WL | 2.83 | 1.77 | 2.19 | 1.12 | 0.5 | 0.95 | 2.78 | 2.37 | 2.71 | 2.33 | 1.92 | 2.07 |
| S1-R-WL | 2.29 | 2.01 | 1.99 | 3.18 | 2.62 | 3.07 | 3.5 | 2.7 | 2.72 | 2.77 | 2.16 | 2.34 |
| S2-L-WL | 2.5 | 1.75 | 2.03 | 2.92 | 2.08 | 2.58 | 3.36 | 3.07 | 3.35 | 4.38 | 3.47 | 5.9 |
| S2-R-Wl | 3.92 | 3.04 | 3.25 | 4.07 | 3.39 | 3.97 | 3.88 | 3.11 | 4.41 | 4.84 | 4.19 | 4.2 |
| S3-L-WL | 4.2 | 3.29 | 3.32 | 4.24 | 3.69 | 4 | 4.24 | 3.3 | 3.96 | 4.4 | 4.2 | 4.39 |
| S3-R-WL | 4.5 | 4.24 | 4.45 | 3.8 | 3.27 | 4.39 | 2.64 | 1.93 | 3.04 | 3.8 | 3.29 | 3.38 |
| S4-L-WL | 2.45 | 1.98 | 2.37 | 4.38 | 3.84 | 5.14 | 1.5 | 1.84 | 1.3 | 1.59 | 1.03 | 1.03 |
| S4-R-WL | 2.14 | 1.54 | 2.19 | 2.04 | 1.88 | 1.66 | 3.37 | 2.16 | 3 | 3.25 | 2.21 | 3.12 |
| S1-L-SL | 0.2 | −0.76 | −0.69 | 1.13 | 0.93 | 1.0 | 2.51 | 1.85 | 1.85 | 3.14 | 3.49 | 3.06 |
| S1-R-SL | 4.32 | 3.64 | 3.87 | 3.13 | 2.15 | 2.72 | 3.92 | 3.52 | 3.52 | 3.9 | 2.92 | 3.78 |
| S2-L-SL | 2.42 | 1.68 | 1.91 | 3.01 | 1.8 | 1.96 | 4.75 | 3.73 | 4.27 | 3.22 | 2.27 | 2.56 |
| S2-R-SL | 5.58 | 5.12 | 5.18 | 4.28 | 3.85 | 4.3 | 4.29 | 3.65 | 4.17 | 4.32 | 4.4 | 4.22 |
| S3-L-SL | 2.08 | 1.72 | 1.72 | 3.59 | 3.03 | 3.06 | 1.83 | 0.85 | 1.63 | 2.34 | 1.27 | 1.86 |
| S3-R-SL | 4.06 | 3.75 | 4.06 | 3.8 | 2.5 | 3.93 | 3.65 | 3.57 | 3.52 | 2.85 | 1.42 | 3.06 |
| S4-L-SL | 4.65 | 4.16 | 4.2 | 3.69 | 3.05 | 3.55 | 4.5 | 3.71 | 4.21 | 2.66 | 1.43 | 1.91 |
| S4-R-SL | 3.08 | 2.67 | 2.91 | 3.74 | 3.44 | 3.35 | 3.76 | 3.21 | 3.29 | 4.7 | 4.09 | 4.63 |
| Mean (N = 16) | 3.20 | 2.60 | 2.81 | 3.26 | 2.63 | 3.10 | 3.41 | 2.79 | 3.18 | 3.41 | 2.74 | 3.22 |
| SD | 1.34 | 1.43 | 1.41 | 1.03 | 1.01 | 1.23 | 0.93 | 0.86 | 0.96 | 0.96 | 1.16 | 1.26 |
| RSD, % | 41.9 | 55.1 | 50.2 | 31.5 | 38.4 | 39.6 | 27.4 | 30.9 | 30.1 | 28.3 | 42.5 | 39.1 |

TABLE 4
Ratio of TEWL of Petrolatum/Titanium Oxide
Formulation to Petrolatum

| | 30 second | | 60 second | | Selected^{b} | |
|---|---|---|---|---|---|---|
| | TEWL | Ratio^{a} | TEWL | Ratio^{a} | TEWL | Ratio^{a} |
| Petrolatum | 3.10 | 1 | 2.45 | 1 | 2.72 | 1 |
| 1% TiO2 | 3.22 | 1.04 | 2.66 | 1.09 | 3.22 | 1.18 |
| 5% TiO2 | 3.16 | 1.02 | 2.56 | 1.04 | 3.06 | 1.13 |
| 10% TiO2 | 3.42 | 1.10 | 2.81 | 1.15 | 3.30 | 1.21 |
| Petrolatum | 3.30 | 1.0 | 2.75 | 1.0 | 2.90 | 1.0 |
| 1% TiO2 | 3.30 | 1.0 | 2.59 | 0.94 | 2.98 | 1.03 |
| 5% TiO2 | 3.65 | 1.11 | 3.01 | 1.09 | 3.31 | 1.14 |
| 10% TiO2 | 3.39 | 1.03 | 2.66 | 0.97 | 3.14 | 1.08 |
| Petrolatum | 3.20 | 1 | 2.60 | 1 | 2.81 | 1 |
| 1% TiO2 | 3.26 | 1.02 | 2.63 | 1.01 | 3.10 | 1.10 |
| 5% TiO2 | 3.41 | 1.07 | 2.79 | 1.07 | 3.18 | 1.13 |
| 10% TiO2 | 3.41 | 1.07 | 2.74 | 1.05 | 3.22 | 1.15 |

We claim:

1. A topical formulation for the treatment of wounds comprising particles selected from the group consisting of titanium dioxide, zinc oxide, talc, and magnesium oxide and a pharmaceutically acceptable petrolatum base, wherein the particles are present in a weight percentage between one and up to 30 percent.

2. The formulation of claim 1, wherein the particles are less than about 100 microns in diameter.

3. The formulation of claim 2, wherein the particles are titanium dioxide.

4. The formulation of claim 3, wherein the concentration of titanium dioxide is between 1 and 20%.

5. The formulation of claim 3, wherein the concentration of titanium dioxide is between 5 and 10%.

6. The formulation of claim 1, wherein the pharmaceutically acceptable base further comprises one or more excipients selected from the group consisting of mineral oils and vegetable oils.

7. The formulation of claim 2, further comprising one or more active agents selected from the group consisting of antibacterial agents, antifungal agents, topical steroids, topical anesthetics, anti-inflammatory agents, and combinations thereof.

8. The formulation of claim 2, further comprising one or more pharmaceutically acceptable excipients.

9. The formulation of claim 1 applied to or impregnated into a bandage, gauze, foam, sponge, and wrap.

10. The formulation of claim 1 in an applicator tube.

11. A method for treating dry skin, wounds or scars comprising administering to a patient in need thereof an effective amount of a topical formulation for the treatment of wounds comprising particles selected from the group consisting of titanium dioxide, zinc oxide, talc, and magnesium oxide and a pharmaceutically acceptable petrolatum base, wherein the particles are present in a weight percentage between one and up to 30 percent.

12. The method of claim 11, wherein the wound to be treated is selected from the group consisting of diabetic ulcers, pressure sores, lacerations, bite wounds, burns, penetrating wounds, surgical wounds, and combinations thereof.

13. The method of claim 11 for the treatment of fine lines and wrinkles, comprising administering to an individual in need thereof an effective amount of the formulation.

14. The method of claim 11 for the treatment of dry skin, comprising administering to an individual in need thereof an effective amount of the formulation.

15. The method of claim 11 for treatment of scars comprising administering to the scar, with or without covering, an effective amount of the formulation for an effective period of time.
